(19) United States
(12) Patent Application Publication
(10) Pub. No.: US 2008/0020368 A1
(43) Pub. Date: Jan. 24, 2008

(54) Method and apparatus for exposing cells to different concentrations of analytes or drugs

(76) Inventors: Mengsu Yang, Kowloon (HK); Cheuk-Wing Li, Chai Wan (HK)

Correspondence Address: Robert K. Tendler, 65 Atlantic Avenue, Boston, MA, 02110, US

(21) Appl. No.: 11/489,849

(22) Filed: Jul. 20, 2006

(51) Int. Cl. C12Q 1/00; C12M 3/00

(52) U.S. Cl. 435/4; 435/287.1

(57) ABSTRACT

A tapered microchannel structure allows individual cells to be reacted with a continuum of concentrations or dosages of an analyte or drug from one sample. A dual sandbag structure that divides up a tapered micro channel into thirds permits performing two simultaneous tests on two different sets of cells isolated in the two sandbags by introducing a single analyte or drug into the region between the two sandbag structures.

### FIELD OF THE INVENTION

This invention relates to an apparatus and method for on-chip monitoring of cellular reactions and more particularly to a method and apparatus for enabling cells isolated in a sandbag structure to be exposed to different concentrations of analyte or drugs in a one-step operation.

### BACKGROUND OF THE INVENTION

As described in an article entitled “PDMS-Based Microfluidic Device With Multi-height Structures Fabricated by Single-step Photolithography Using Printed Circuit Board as Masters” by Cheuk-Wing Li, Chung Nam Cheung, Jun Yang, Chi Hung Tzang and Mengsu Yang, published in the Royal Society of Chemistry, July 2003, a rectilinear microfluidic device formed photolithographically on a printed circuit board provided for the interaction of two fluids introduced into a microchannel so that the two laminar fluid flows interdiffuse. One of the fluids included individual cells that were isolated and entrapped at known locations in a multi-height sandbag structure. The trapped cells interacted with fluid that included either an analyte or drug composition, with the cell reaction read out using luminescent fluorescence techniques.

Note that on-chip monitoring of cellular reactions is disclosed in Patent Application Publication No. US2003/0175944A1 by Mengsu Yang et al. Moreover, absorption-enhanced differential extraction using laminar flow and an extraction channel is shown in U.S. Pat. Nos. 5,971,158 and 6,200,814.

As will be appreciated, a microfluidic device may be used in a number of applications to expose cells to different analytes, drugs or other assay fluids in which two fluids laminarly interact for cell biologic studies including drug screening, diagnosis and any situation where one needs to generate a reaction between molecules in a fluid and cells.

While the laminar flow microchannel device described in the above article is extremely useful in the cell interactions, in order to vary the concentrations, drug doses or the like, one has to stop the process and then introduce a different concentration or dosage into the microfluidic device.

This type of fluidic device, while useful as a diagnostic tool, involves batch processing. Batch processing is time-consuming, especially considering diagnostic tests where one seeks, for instance, to interact even-increasing concentrations of saline with cells to ascertain when, for instance, a red blood cell breaks apart.

Moreover, such batch testing techniques are not optimal for drug screening processes in which drug companies screen hundreds of thousands or millions of compounds or molecules with a variety of concentrations or dosages of reactants.

Moreover, in most cases the drug companies have available only extremely small samples. It is a quite common screening practice to divide up the relatively small sample into further smaller samples and test each of the smaller samples with different concentrations or dosages. As will be appreciated, such protocols rapidly deplete the supply of the sample.

Thus there is a necessity to provide a way of testing cells against many different analyte concentrations or drug dosages in a single-step process. This would avoid having to provide multiple samples of differing concentrations or dosages.

Moreover, it would be useful to be able to run simultaneous and parallel tests, for instance between drugs and placebos or between diseased cells and normal cells, so that the results could be immediately read out for each concentration or drug dosage.

If such could be accomplished, the testing or processing times would be dramatically reduced due to the fact that one would not have to separately prepare different concentrations or dosages and run separate tests. If one could subject the cells to a continuum of concentrations or dosages, one could rapidly ascertain the biologic activity of analytes or drugs in varying concentrations and/or dosages either on single or multiple cell types; or even the effect of differing dosages of a drug versus a simultaneously administered placebo.

By way of further background, from the inherent scalability of photolithography, microfluidic technology improves efficiency and replication of experimental procedures to improve throughput. During throughput increment, the accompanying control and spatial resource consumption becomes an increasing concern. It will be shown that by a unique arrangement in channel geometry, laminar flow may be manipulated to maximize throughput at minimal resource consumption to provide a high throughput-over-resource (high T/R) microdevice.

Although it has been shown that it is possible to increase throughput by parallel processing of repetitive functional units, it consumes more spatial area and aggravates the control complexity of a microdevice. Previously, this control complexity issue has been attenuated by outlet vial sharing to sustain throughput. Although current technology enables high-density design repetition, vial sharing alone is not effective enough to restrain the increasing control complexity. To tackle prominent controlling issues associated with large-scale integration, repetitive chambers have been controlled by fluidic multiplexors through a network capable of driving N number of fluid channels with only 2log₂ N of control resources. Although it is possible to individually address 256 chambers using this strategy, the latency in sequentially addressing all chambers increases linearly with the number of chambers. In another recent study, a multi-layer PDMS microdevice provided with 17 control resources (plus 9 solution vials and 54 movable valves) was capable of increasing throughput with no increment in control complexity. Simultaneous processing of 3 bacterial sample dilutions (from a single source) was performed in hardwired microchannels integrated with lysing and DNA extraction functionalities. Although the control resources did not increase with repetitive units arranged in one linear direction, the scalability was hindered by the spatial design consumption.

Therefore, control and spatial refinements that accompany throughput increment are critical obstructions to the future development of microfluidic devices.

### SUMMARY OF INVENTION

It has been found that one can obtain a continuum of concentrations or dosages from low to high by tapering the laminar flow interaction channel such that as the two laminar liquid streams flow from a wide inlet to a narrowed outlet the diffusion between the two flows increases as the two fluid flows are forced together by the narrowing walls of the micro channel. Thus rather than having the majority of the diffusion occurring at the inlet end of a straight micro channel where equilibrium is quickly reached corresponding to one concentration or dosage to which the cells in the sandbag are subjected, in the subject invention there is decreased diffusion at the inlet end and an increased diffusion at the outlet end to provide a diffusion continuum. This in turn provides a continually increasing concentration or dosage level as the two streams are squeezed together by the tapered micro channel.

Since the increased concentrations or dosage levels occur at different positions relative to the sandbag structure, the individual cells at different locations in the sandbag structure are subjected to different concentrations of analytes or different dosages of a drug.

When the analyte or drug cell interaction is read out at the different positions on the sandbag structure, the reaction at the different positions can be correlated to the specific concentration or dosage at that position.

The result is that one can position cells from a fluid stream at different positions along the sandbag structure where they are immobilized. One then reacts the individual cells at the different positions with different concentrations due to the different and increasing diffusions as one proceeds toward the outlet end of the micro channel structure.

All this is accomplished with only one concentration or dosage of analyte or drug at the inlet port, with the different concentrations or dosages supplied by the narrowing microchannel structure.

This permits testing at a virtual continuum of concentrations or dosages from one sample without having to prepare different samples at different concentrations or dosages.

Note, the increasing diffusions with distance from the inlet define a concentration gradient. This gradient corresponds to the difference in micro channel width from the inlet to the outlet, with the variation in width itself constituting a dimensional gradient. The larger the dimensional gradient, the longer is the region on the sandbag structure subjected to discernibly different concentrations.

In one embodiment the microchannel device is divided into thirds, with two linear sandbag structures lining the interior walls of the dividers between the three micro channel chambers. These two sandbag structures are separated at the inlet end and tapered together through the use of the microchannel structure towards the common outlet.

This dual sandbag structure permits simultaneous testing of for instance normal cells and diseased or non-normal cells with the same analyte and at the same concentrations or dosages. This permits one to immediately compare the interaction of two cell types with the same sample and the same concentrations or dosages.

Moreover, adjusting the fluid pressures can determine what side of the sandbag structure the cells lie on. This permits for instance injecting the drug into the middle channel and testing for instance normal cells on the inside of one sandbag structure with the drug, while simultaneously testing cancer cells on the inside of the other sandbag structure with the same drug and at the same dosage.

Thus one can expose two different types of cells to one sample. Since the micro channel structure is tapered, the above test can be made at a continuum of concentrations or dosages.

Pressure control and the dual sandbag structure permits simultaneously running two parallel experiments.

Note that by simply altering channel geometry, the subject invention shows compact integration of scalable functionalities (cell immobilization and gradient generation) to permit cell-based biological assays without the need for complicated controls. By manipulating laminar flow inside the channels, the subject design is capable of processing about 200 single cells with in-situ sample dispensing, mixing and dilution within an effective design area of 0.2×0.2 cm². Another important feature is the ability to adjust the concentration gradient profile at low fluidic flow rates using passive fluidic controls, thus to improve the compatibility with a variety of instruments commonly available in the laboratory.

In summary, a tapered microchannel structure allows individual cells to be reacted with a continuum of concentrations or dosages of an analyte or drug from one sample. A dual sandbag structure that divides up a tapered micro channel into thirds permits performing two simultaneous tests on two different sets of cells isolated in the two sandbags by introducing a single analyte or drug into the region between the two sandbag structures.

### BRIEF DESCRIPTION OF THE DRAWINGS

These and other features of the subject invention will be better understood in connection with a Detailed Description, in conjunction with the Drawings, of which:

FIG. 1 is a diagrammatic illustration of the subject microchannel microfluidic chip module indicating that a predetermined volume at the inlet to the microchannel can control the flow stream and thus the microchannel output pressure;

FIG. 2 is a diagrammatic illustration of the subject tapered microchannel showing controlled vial volumes of liquid at various inlet orifices to the microchannel, the vial volumes controlling cell docking and analyte or drug reactions with docked cells;

FIG. 3 is a diagrammatic illustration of a three-cavity, tapered microchannel module as seen from the top thereof, illustrating two tapered outlying chambers and a tapered interior chamber, with the outlying chambers lined on either side with a sandbag structure for capturing cells flowing from respective chamber inlets to the outlet under the control of a docking component whose flow velocity and thus pressure is controlled by the associated vial volume;

FIG. 4 is a diagrammatic illustration of the sandbag structure of FIG. 3, illustrating flow through microchannels between sandbag elements;

FIGS. 5A and 5B are diagrammatic illustrations of the docking of cells introduced into one of the outlying chambers of the microchannel structure of FIG. 2 as the cells move to lie against and be captured by the sandbag structure so that individual cells are docked to the interior of the sandbag structure;

FIG. 6 is a diagrammatic illustration of flow velocities in a channel indicating faster flow velocities in the middle of a channel, with such action removing excessive cells that are built up over docked cells, leaving single cells trapped by the sandbag structure;

FIG. 7 is a diagrammatic illustration of docked cells in the structure of FIG. 2 to be reacted with an analyte or drug introduced into the two outlying chambers;

FIG. 8A is a diagrammatic illustration of a method of achieving a gradient concentration in one of the two outlying chambers of the microchannel module of FIG. 3;

FIG. 8B is a diagrammatic illustration showing by the shading, the gradient concentration in one of the outlying chambers;

FIG. 9 is a table illustrating a two cell-line operation in which solutions with the cells are introduced simultaneously into the two outlying chambers, with cells being reacted with the same analyte or drug;

FIGS. 10A-10H are a diagrammatic representation of the process used to create a sandbag structure having microchannels;

FIG. 11 is a diagrammatic illustration of the diffusion reaction length in the subject tapered channel having been provided with a 15° taper;

FIG. 12 is a diagrammatic illustration of a prior art non-tapered T-channel indicating a constant diffusion distance;

FIG. 13 is a photomicrograph of the dispersion between two fluids at the inlet of the tapered channel of FIG. 11, indicating a diffusion gradient due to the tapered construction of the channel;

FIG. 14 is a photomicrograph of the diffusion in a non-tapered T-channel indicating all of the diffusion occurring at the inlet port;

FIG. 15 is a graph of normalized fluorescence intensity versus distance showing a tapered gradient for the tapered channel versus essentially no gradient for the non-tapered channel;

FIG. 16 is a diagrammatic illustration of tapered channels with different angles of taper, as well as a diagrammatic illustration of a non-tapered T-channel;

FIG. 17 is a graph of normalized fluorescence intensity versus sandbag positions, showing the different gradients for different taper angles for the tapered channel, as well as a flat or non-existent gradient for the non-tapered T-channel;

FIG. 18 is a photomicrograph of cell docking along one sandbag structure in a dual sandbag triple chamber embodiment of the subject invention;

FIG. 19 is a photomicrograph of cell docking in a non-tapered T-channel;

FIG. 20 is a graph of docking potential versus sandbag position for the tapered channel, indicating a predictable flat docking and gradient;

FIG. 21 is a graph showing docking potential versus sandbag position for a non-tapered channel, indicating the variability in docking potential versus sandbag position;

FIG. 22 is a structural model when both gradient and docking components are of I unit radii. The pressure drop is linear along the length of microchannels;

FIG. 23 is a modified structural model (as described in FIG. 22) where both gradient and docking components are enlarged to 2 unit radii. The pressure drop along each channel is insignificant so that a stable pressure difference between two channels is established;

FIG. 24 is a photomicrograph indicating phosphorescent returns when probing the cells in a sandbag structure with green light, with red light, and with a combination, indicating for the combined case cell densities for the various positions from the rear of the sandbag structure to the front of the sandbag structure, indicating that the microchip is capable of interrogating the responses of two dyes (drug) under two differing continuum of dosages;

FIG. 25 is a graph of normalized fluorescence intensity versus sandbag position for the theoretical TMR profile and the theoretical CAM profile, along with response profiles for each, indicating that the differing continuum of dosages between the two dyes contribute to the red, TMR dominant staining at the sandbag front;

FIG. 26 is a diagrammatic illustration of the CAM dose response for a line of cells docked on a sandbag from 368 s to 602 s., showing that along the sandbag, one can retrieve the dose response of all cells at a certain moment while one can retrieve the time course response of every individual cell by tracking the responses over time in which 5 images=5 time points;

FIG. 27 is a three-dimensional graph of sandbag position versus time versus fluorescent intensity, showing the CAM dose response at various positions and times;

FIG. 28 is a graph of fluorescent intensity versus time, illustrating that the time course CAM response is linear over time under various concentrations;

FIG. 29 is a graph of normalized total esterase activities versus CAM concentration for the tapered channel experiment of FIGS. 2-9, and a control experiment performed on a conventional microplate reader, illustrating that the results retrieved from the subject microchip are comparable to those associated with a conventional instrument;

FIG. 30 is a composite of two photomicrographs showing the test of a photodynamic drug candidate, TMR, in which both upper and lower sandbags of a taper microchip device were docked with cells and laser irradiation was exclusively applied to the cells enclosed in the black dotted framework, with the response to TMR recorded in the bottom confocal image with corresponding plot in FIG. 31; and, FIG. 31 is a graph of fluorescence intensity versus TMR concentration for the experiment of FIG. 30, showing that the 3.73 μM concentration at 50% intensity was required to trigger enhance the fluorescence response for the enclosed cells that were simultaneously irradiated with laser.

### DETAILED DESCRIPTION

From the above it will be appreciated that the testing procedure involving the tapered microchannels involves first loading the cells onto the sandbag structure by a cell docking operation mode. Once the cells are in place, one switches to an analysis mode, which in one embodiment involves the use of a Calcein-AM (CAM) dye in which one reacts the dye/drug combination with the cells that are trapped in the sandbag.

As will be appreciated, docking the cells follows Bernoulli's equation such that the flow Z, which is zero at the top of a liquid in a container, flows at a rate through the orifice as a function of the liquid volume above the orifice. This principle permits control of the cell during docking procedure to be described by controlling the volume in the vials of liquids that introduce into the microchannel module to be described. This means that just the right pressure can be exerted on the cells to lodge individual cells into a sandbag structure.

Referring to FIG. 1, Bernoulli's equation applies in the subject invention in which, in cross-section, a microchip module 20 includes an inlet 22 between a top portion 24 of the microchannel and a base 26, wherein liquid 28 passes through inlet 22 towards outlet 30. Here the two sections formed in the microfluidic chip module are shown by base 26 and top portion 24 that is channeled using a PDMS channeling process.

In this instance the pressure at outlet 30 is determined by the inlet volume. Thus channel pressure is determined by inlet volume.

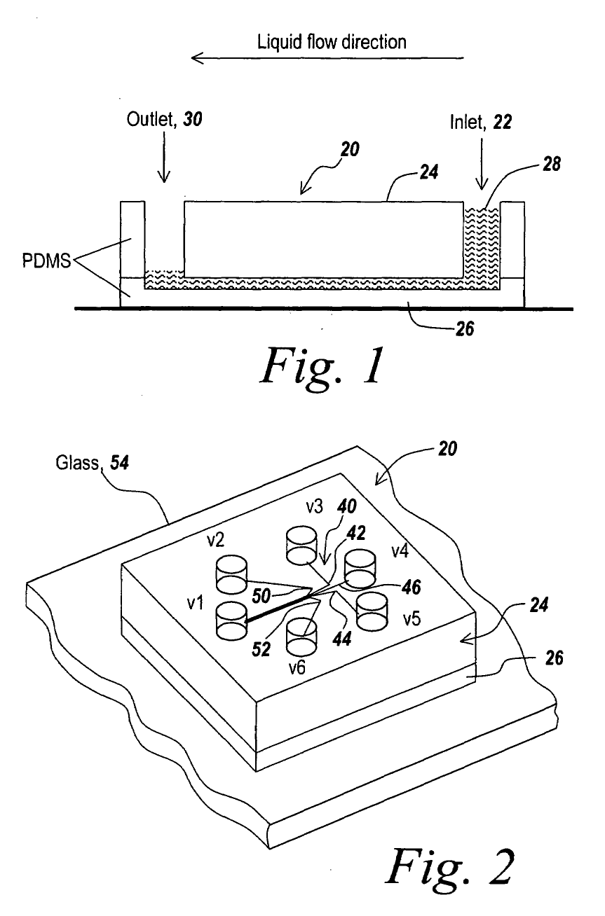

Referring now to FIG. 2, a microchip channeled module 20 composed of base 26 and channeled top portion 24 is provided with a microchannel structure 40 in which a central channel is formed by a channel 46 fed by vial v4. Channels 42 and 50 are fed by vials v3 and v2, while channels 44 and 52 are fed by vials v5 and v6 respectively. The outlet port is a vial v1, which constitutes the outlet port for the subject device.

The entire structure may be mounted on a glass base 54, with portions 24 and 26 of the microchannel structure being formed by PDMS techniques.

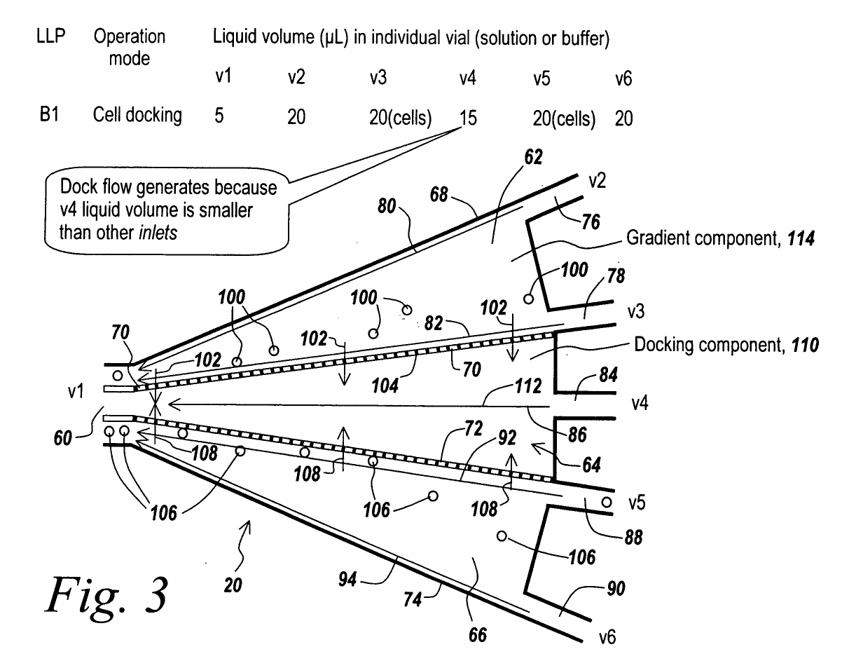

As seen in FIG. 3, the microchannel module has an outlet port 60 corresponding to v1 and in one embodiment has three chambers 62, 64 and 66 formed respectively by wall 68, sandbag structure 70, sandbag structure 72 and wall 74.

The tapered structure is clearly visible by the sloping of walls 70 and 74 as well as the sloping sandbag structures 70 and 72 tapered towards the narrowed outlet 60.

In one embodiment, left chamber 62 is provided with inlet orifices 76 and 78 to be able to introduce various liquids from vials v2 and v3 so as to flow as indicated by arrows 80 and 82.

For central chamber 64, one has an inlet orifice 84 to which vial v4 is coupled, which results in a liquid flow 86 towards exit orifice or outlet 60.

The right chamber 66 is provided with inlet orifices 88 and 90, to which vials v5 and v6 are coupled to form liquid flows 92 and 94 as indicated by the associated arrows.

In a docking step, with respect to chambers 62 and 66, various flows characterized by flow velocities and pressures are used to carry cells and to embed or entrap individual cells 100 in sandbag structure 70. The flow directions are indicated by arrows 80 and 82. Because of the tapering there is lateral flow as indicated by arrows 102 through the sandbag structure. This lateral flow serves to capture or trap individual cells in individual microtunnels in the sandbag structure, one of which is shown at 104 for left chamber 62.

Likewise, for right chamber 66, individual cells 106 are trapped within the microtunnels of sandbag structure 72 by virtue of the pressure exerted by flows 92 and 94 so as to embed cells 106 at various locations in the sandbag structure 72. Here again, a lateral flow as shown by arrow 108 causes the trapping of cells 106 in sandbag structure 72.

How the lateral flow rate and pressure are precisely controlled involves the vial volumes, both at the inlets to the left and right chambers and the vial volume for the central chamber. With respect to the control of the docking, a docking component 110 is introduced into inlet orifice 84 to establish the flow indicated by arrow 112 via the vial v4 volume, such that the flow indicated by arrow 112 counters the lateral flow associated with arrows 102 and 108, so as to prevent the passage of the individual cells completely through the sandbag structure.

It will be appreciated that with the gradient component illustrated at 114 is at one velocity and thus one pressure. However, the docking component is at a different velocity and pressure controlling the velocities and pressure controls the trapping of the cells in the sandbag structure, which is done by adjustment of the volumes v2-v6.

At the top of FIG. 3 is shown a table in which the liquid volume in microliters for an individual vial of solution or buffer indicates that for a given liquid volume a certain cell docking result occurs.

Thus, in the illustrated example, the particular liquid level program LLP represents a specific combination of liquid volumes in microliters among the six vials, the purpose of which is to initiate cell docking.

Note that the vial volume can be changed by sucking out liquid or adding liquid by pipette to establish minute amounts of liquid, e.g., from 1 to 20 microliters.

Thus, for instance, the input can be controlled by having v1 have a 5-microliter output, whereas v2 establishes a 20-microliter input for one of the gradient components for the left chamber, v3 a 20-microliter input for another of the gradient compounds for the left chamber, v4 a 15-microliter input for the docking component, v5 a 20-microliter input for a different gradient component, this one for the right chamber, and v6 a 20-microliter input for the second gradient component for the left chamber.

Note that vials v3 and v5 include solutions having cells, the cells of which are to be docked at the corresponding surfaces of respective sandbag structures.

It can be thus seen by the adjustment of the volume in a vial that one can effectuate pressures across each of the two sandbag structures to trap or capture cells to the inside of the particular sandbag structure during the cell docking operation.

Note that the vial v4 has a 15-microliter volume to generate a counteracting docking flow lower than that of the lateral gradient flows because the v4 liquid volume is smaller than that associated with the other inlets.

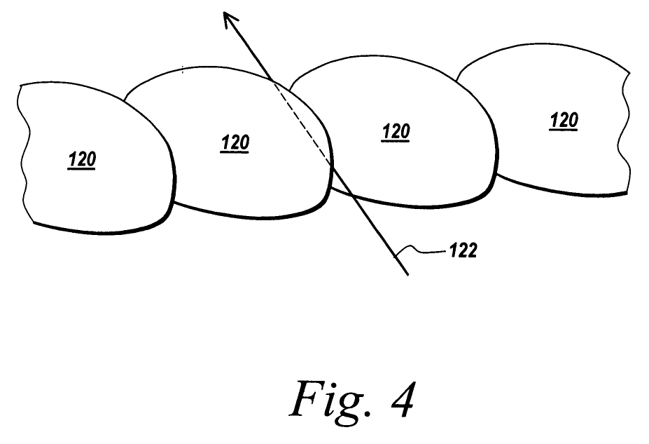

Referring to FIG. 4, the sandbag structure is shown in which individual dome-shaped elements 120 constitute the aforementioned sandbag structure. These elements have touching edges but which nonetheless define a dam over which flow indicated by arrow 122 is permitted. The sandbag structure itself forms microtunnels between adjacent sandbag elements so that while flow is allowed to occur between the sandbag elements, cells are of a size that will be trapped between the sandbag elements and will exist to one side of the sandbag structure or dam depending on the flow in the microtunnels between the sandbag elements.

It is therefore possible with the proper adjustment of volumes that one can trap individual cells in the microtunnels between the sandbags so that they can exist on the interior portion of the particular microchannels, meaning the portion that faces away from the central chamber.

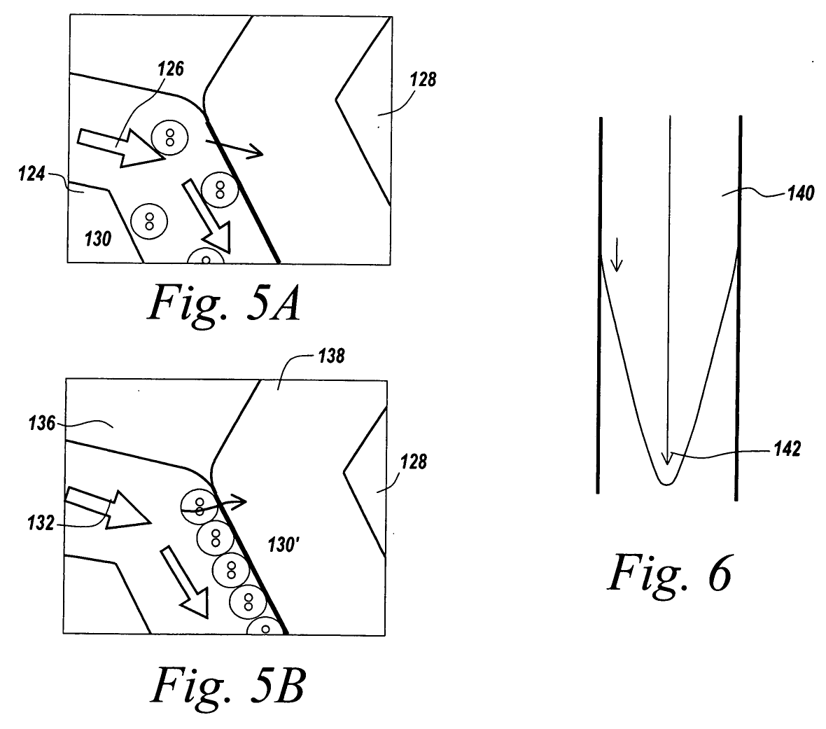

Referring to FIG. 5A and FIG. 5B, individual cells are introduced into an inlet 124 so as to flow in the direction of arrow 126 towards a sandbag structure 128, with the individual cells being illustrated at 130. As can be seen in FIG. 5B, cells 130′ move towards sandbag structure 128 by virtue of flow 132, such that the cells line up along the interior wall of the sandbag structure. Note that the fluid flow during cell docking is such that liquid pressure in microchannel 136 is higher than that associated with microchannel 138, such that a fraction of the fluid flows through or over the sandbag structure dam from channel 136 into channel 138. Thus the hydrodynamic pressure difference brings the cells in channel 136 to dock along the sandbag structure dam.

After complete cell docking, the flow across the sandbag structure dam is reduced so that excessive cells become more difficult to approach the laminas of slower velocities near the dam or sandbag structure and are driven away along the main flow route.

The result is a line of single cells that are docked along the sandbag structure dam without a multi-layer formation.

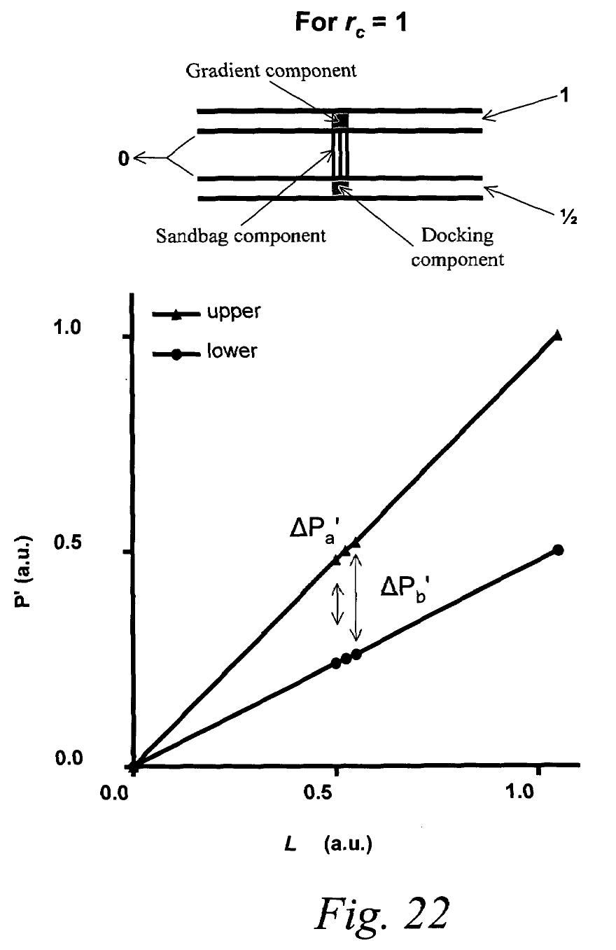

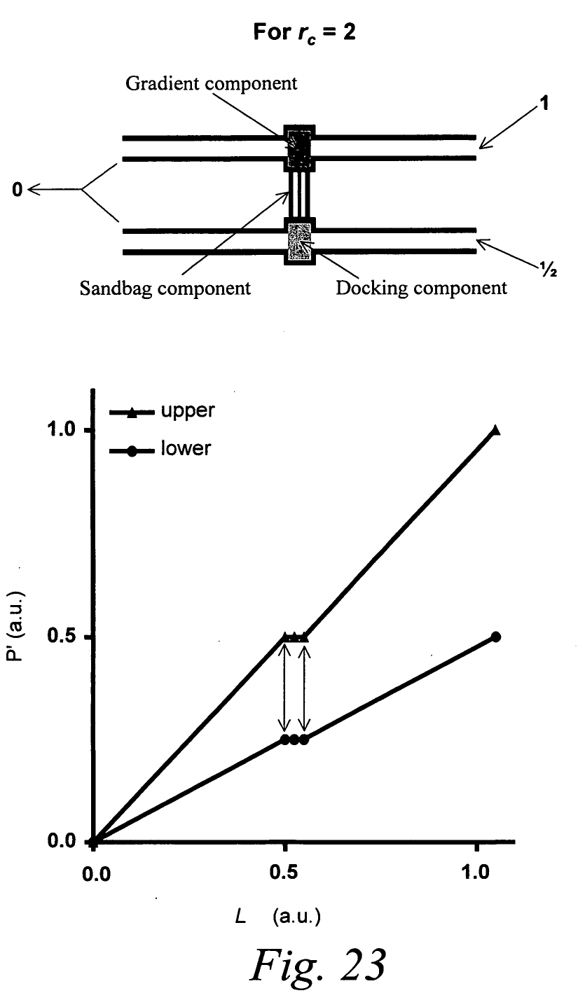

In an attempt to obtain a pressure gradient, initially a structural model was introduced comprising upper and lower microchannels interconnected with a “sandbag-like” structure, sandbag component (FIG. 22). The two channels were of circular cross-sectional dimension and unit length. The sandbag components, positioned at the midway of the channel system, consisted of microtunnels that allowed cells to dock individually at positions along the channel. Gradient and docking components were introduced into two channels adjacent to the sandbag component. In order to illustrate how one could obtain a pressure and velocity gradient the effect of geometric modulation was investigated. The geometric modulation included introducing a partial enlargement by changing channel radii of gradient and docking components, r_{c} (FIG. 23). In both models, applied pressure was 1-fold higher to the upper (1 unit pressure) than the lower channel inlet (0.5 unit pressure) while flows were converged to a common outlet at zero pressure. Thus, a right-to-left fluid flow and a pressure difference were generated along and between the two channels respectively. The sandbag component was assumed to have high fluidic resistance such that negligible flow could pass through. According to above conditions, cell docking that was driven by pressure profiles of the two channels can be derived from the first principle of fluid dynamics,

$$Δ {P}^{′} = \frac{Q}{{r}^{4}} L \qquad ( 1 )$$

where ΔP′=pressure drop along channel divided by a constant, Q=flow rate, L=channel length and r=channel radius. Charting P versus L, consider the ΔPₐ′ (ΔP′ along gradient or docking component) and ΔP_{b}′ (ΔP′ between gradient and docking component) values.

In accordance with Equation 1, increasing radius could proportionally reduce the ΔPₐ′ values, given that the partially enlarged gradient and docking components were short enough to maintain the overall flow rate constant. It was found that the ΔP_{b}′ values decreased less steeply for r_{c}=2 than for r_{c}=1 along fluidic flow direction. A stable and constant pressure difference was achieved by modulating the slope of pressure profiles with a 1-fold radii increment at the gradient and docking components. This simple geometric modulation suggested the possibility of maintaining a constant ΔP_{b}′ that is important to immobilize a maximum number of individual cells along a sandbag structure of fixed length.

Note liquid pressure is dropping along the microchannel of the gradient component, so no one pressure exists.

Although enlarging channel radii, r_{c} can create one pressure and thus one velocity across the sandbag, gradient concentrations generated by merely enlarging the microchannels did not prove effective. This led to the tapered channel shape of the subject invention. Corresponding to this tapered shape, when the pressure profile (interrogated from the front to the rear of the upper sandbag structure) is plotted, the ΔP_{b} pressure between gradient and docking component is not constant but rather varies with the taper. So too are the velocities tapered.

Comparing with traditional T-shaped channel geometry, the tapered channel geometry increased gradient concentrations and provided a more stable ΔP_{b}, noting the more stable the ΔP_{b}, the more biological cells that can be immobilized on the sandbag structure.

Referring to FIG. 6, the reason that excessive cells are removed after full docking is the fact that, for instance, in a microchannel 140, the flow velocity 142 is faster in the middle of the channel such that, as viewed from the top of the microchannel, the flow in any event is less at its extremities. This results in the carrying away of excessive cells so that only single cells are trapped within the sandbag structure.

Referring now to FIG. 7, microchannel module 20 comprising the three chambers 62, 64 and 66, is shown within individual cells trapped in respective sandbag structures 70 and 72 such that these structures hold individual cells 100 and 106.

In order to provide for the continuum of dosages or concentrations, individual cells 100 and 106 are subjected to gradients 150 and 160, which may include analyte or dosages and dyes such that, with the appropriate volumes at v2, v3, v4, v5 and v6, cells 100 and 106 will be reacted with a gradient density of solutions introduced at v2 and v3 as well as at v5 and v6, with the volume at v4 controlling the rate of penetration of the gradient solutions as illustrated by arrows 162 and 164. The result is the reaction of the cells docked at the sandbag structures with a continuum of analyte or dosage levels.

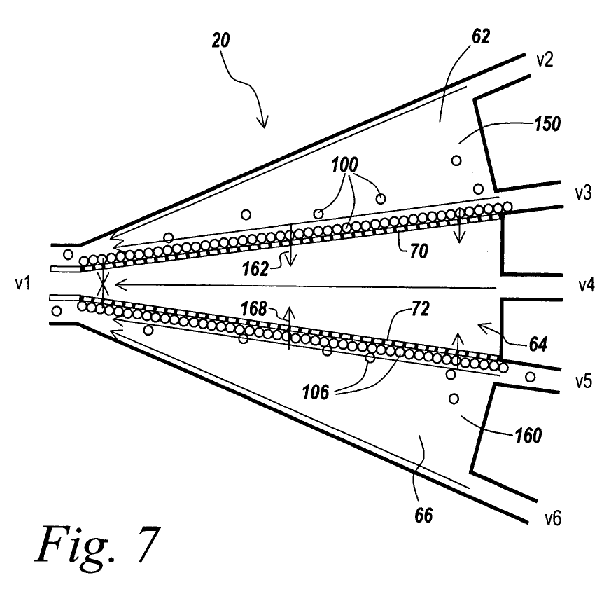

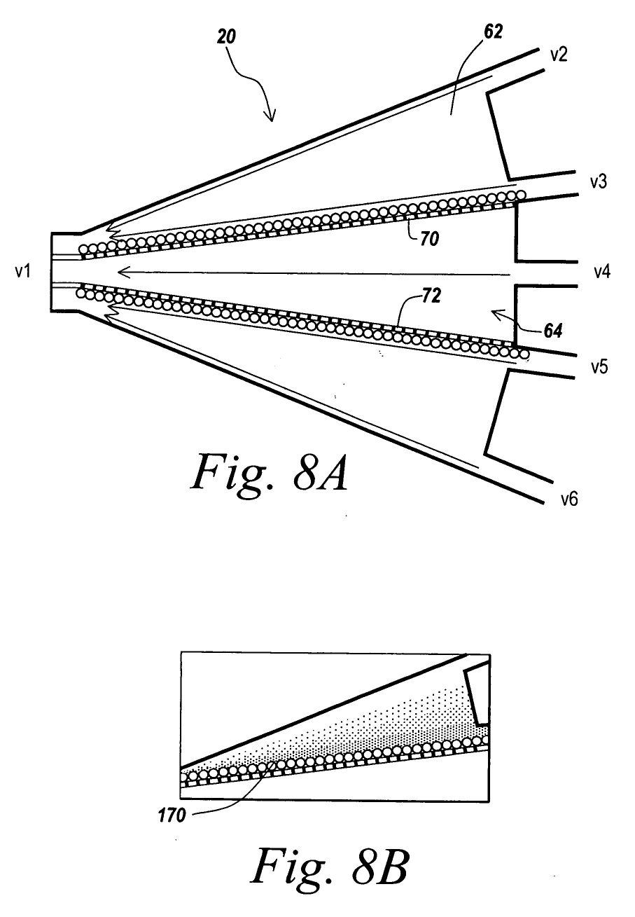

In one embodiment, the docked cells are reacted with the analytes or drug concentrations using suitable solutions and buffers such that, as illustrated in FIG. 8A, the cells on the interior of sandbag structures 70 and 72 are reacted with a gradient of solution as illustrated in FIG. 8B, with the shading at 170 illustrating the change in concentration or density along chamber 62.

In order to achieve this density gradient, the volume at v1 is 5 microliters where at v2 the solution with the dye is at 17 microliters, with v3 also being at 17 microliters as well as v4 and v5 being at 17 microliters. Note the volume at v6 is also at 17 microliters.

Vial v2 carries the dye, whereas the vial v3 carries a buffer. Thus the reactive component or solution is introduced by vial v2, whereas the volumes at the other inlets are kept constant so that there is no penetration of the liquid in left chamber 62 into central chamber 64.

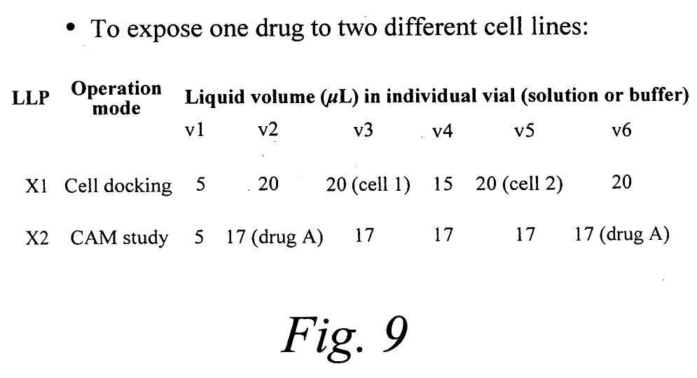

Referring now to FIG. 9, a table is introduced to indicate how one might expose one drug to two different cell lines. This can be done by adjusting the contents and the volumes in vials v1-v6 as illustrated. In this case, Cell Line 1 is introduced by vial v3 during a cell docking operation in which the v3 volume is 20 microliters, whereas Cell Line 2 is introduced by vial v5, again at 20 microliters. Note the volume at v4 is 15 microliters so as to permit docking flow as illustrated previously. The volumes at vials v2 and v6 are likewise 20 microliters during this cell docking operation. More particularly, what is presented is a table illustrating how it is possible to expose one drug to two different cell lines. As can be seen, vial v3 is used to dock a first cell line, whereas vial v5 is used to dock a second cell line. Note, as can be seen from FIGS. 8A and 8B, that the first cell line is docked in chamber 62, whereas the second cell line is docked in chamber 66.

During the CAM study, the particular drug to which both cell lines are to be reacted is introduced at vials v2 and v6, with all volumes being equal, v2-v6, so that the flow goes by the cells carried interiorly of each of the chambers 62 and 66 and not into chamber 64.

Thus, two different cell lines can be tested simultaneously with a given drug without having to vary the concentrations of the drug or drug dosage levels in a batch process, since a continuum of drug dosage levels will be interacting with Cell Lines 1 and 2. Optical readout of the dye concentrations indicates the reaction of the cells to the continuum of dosages of the drug so that immediate comparisons can be made in a continuous flow process as opposed to a batch process, where one has to wait for two sequential batches to finish.

It can therefore be seen that the time involved in testing drugs and analytes on particular cell lines is dramatically reduced over batch processing times due to the continuous flow technique made possible by the tapered microchannel structure and the vial volume control of the process.

It is important to note that the vial volumes themselves provide for the controlled flow rates and differential flows so that the entire process can be quite readily controlled by vial volume itself.

More particularly, to realize a microdevice unit with high T/R ratio, it is possible to simultaneously maximize throughput together and minimize expenses in fabrication, control and spatial resources. To maximize throughput in cell-based microfluidics, the scalability among three functionalities is concerned: 1) cell localization structures, 2) gradient coverage and 3) a method to distribute cells on the localization structures.

Firstly, as the overall throughput is proportional to the total number of biological cells simultaneously handled by the device, it is important in choosing a localization component that scales up easily to accommodate more cells. The subject sandbag component is easy to fabricate by PCB microfabrication processes and scales up over distance without collapse.

Secondly, the distance of concentration gradient coverage affects the overall throughput. As the geometry of microchannel dictates the diffusion dimension, concentration profiles are generated by the V-shaped channels to demonstrate the scalability of gradient coverage at slow fluidic flow rates.

Thirdly, the ability to distribute cells on extended localization structures such as the sandbag structure is of equal importance to the overall throughput. Cell docking, a functionality only achievable by integrating sandbag, gradient and docking components, is employed for cell distribution. As laminar flow across the sandbag component is varied by the geometries of the gradient and docking components, the subject unit with V-shaped channels illustrates the scalability of cell the docking accomplished by the subject invention.

No matter how the individual functionalities are scaled up, the overall throughput is still limited by the one with the smallest scalability. Therefore, it is critical to consider the regularity among scalable functionalities to achieve maximal overall throughput in the subject unit. It will be appreciated that the microfabrication of the sandbag component, gradient coverage and docking capacity are limiting factors to the overall throughput.

Throughput increment strategies should be implemented at minimal resource to realize a high throughput-over-resource (T/R) microdevice unit. The PCB microfabrication described here can generate a multi-height structure in a single round of photolithography while expensive chrome masks, that require at least two masks to make a conventional dam or weir, are not used. Without using hardwired, virtually separated channels, one can induce slow laminar flow inside a microchannel by the subject tapered geometry to distribute the analyte and alter its coverage over a compact spatial area, in one embodiment less than 0.2×0.05 cm². Likewise, instead of using individual chambers, one manages to align multiple single cells along designated sandbag positions by controlling the ubiquitous laminar flow with different channel geometries. To further reduce spatial extent, the gradient coverage and cell docking functionalities shares a common design area. Since independent to the number of hardwired channels/chambers, the throughput of this microdevice can increase in a compact area without the need to employ complicated controls.

### EXAMPLES

TABLE 1
Liquid level program (LLP) used in fluid manipulations

| | | Liquid volume in individual vial (μL) | | | | | |
|---|---|---|---|---|---|---|---|
| | Operation | (solution or buffer) | | | | | |
| LLP | mode | V1 | V2 | V3 | V4 | V5 | V6 |
| A1 | Dye gradient | 5^{a} | 17(EB)^{b} | 17^{a} | 17^{a} | 17^{a} | 17^{a} |
| A2 | Dye filled | 5^{a} | 17(EB)^{b} | 17(EB)^{b} | 17(EB)^{b} | 17(EB)^{b} | 17(EB)^{b} |
| B1 | Cell docking | 5^{c} | 20^{c} | 20(cells)^{d} | 15^{c} | 20(cells)^{d} | 20^{c} |
| B2 | On-chip CAM | 5^{a} | 17(CAM)^{e} | 17(CAM)^{e} | 17(CAM)^{e} | 17(CAM)^{e} | 17(CAM)^{e} |
| | staining | | | | | | |
| C | TMR + CAM | 5^{a} | 17(TMR)^{g} | 17^{a} | 17^{a} | 17^{a} | 17(T_C)^{h} |
| | study | | | | | | |
| D | CAM study | 5^{a} | 17(CAM)^{f} | 17^{a} | 17^{a} | 17^{a} | 17^{a} |
| E | TMR study | 5^{a} | 17(TMR)^{g} | 17^{a} | 17^{a} | 17^{a} | 17(TMR)^{g} |

^{a}HstCa-1
^{b}EB: ethidium bromide, 25 mM
^{c}HstCa-2
^{d}cells: HL-60 cells, cell density 5 * 10⁶
^{e}CAM: Calcein-AM, 10 μM
^{f}CAM: Calcein-AM, 50 μM
^{g}TMR: Tetramethylrosamine, 10 μM
^{h}T_C: Mixture of Tetramethylrosamine and Calcein-AM, each 10 μM

As to laminar flow control by channel geometry, its effect on gradient coverage scalability is now described.

For syringe pump driven microfluidics at a typical average flow speed (˜1000 μm/s), diffusive mixing is inefficient and requires long mixing length (>1×10⁴ sum) for homogenization as seen by the following equation:

$$Mixing length = U \frac{{I}^{2}}{D} \qquad ( 1 )$$

where U=average flow speed, I=cross-sectional dimension and D=molecular diffusivity. The concentration gradient distribution within a microchannel is given by

$$C ( t , x ) = \frac{1}{2} {C}_{0} {\sum }_{n = - ∞}^{∞} \{ erf \frac{h + 2 nw - x}{2 \sqrt{Dt}} + e rf \frac{h - 2 nw + x}{2 \sqrt{Dt}} \} \qquad ( 2 )$$

where C (t, x) is the concentration at time t and at point x, w the width of channel, and C₀ the concentration before intersection. h is the width of the initial distribution.

To manifest rapid mixing in this regime, a number of structures have been proposed, e.g., a chaotic staggered herringbone mixer, staggered vertical pillars, and a microchannel packed with micro-beads or chemically patterned surface patches. On the contrary, very limited research has been dedicated to the opposite challenge: lengthening gradient coverage at slow average flow speed (˜50 μm/s), where diffusive mixing becomes very efficient. In the subject invention, the novel microfluidic channel geometry addresses this challenge and leverages the advantages of passive microfluidic operation.

For a layout for two types of gradient components, V15 and T90 within microdevice units, V15 and T90 were given by the merged shape and angle of the inlet channel midlines. Along the interrogation lines aside both gradient components, ethidium bromide (EB, with diffusion coefficient of 4.15×10⁻¹⁰ m²s⁻¹) gradient profiles were simulated. Essentially equivalent to a typical T intersection, the gradient coverage scalability of T90 was obviously lower than V15 by showing a gradient plateau from 0 to 1650 μm. In contrast, long gradient coverage of V15 properly utilizes the length of the entire microchannel assigned for the sandbag component, the structure for cell localization or docking. Moreover, the gradient coverage of V-shaped models was controllable by altering the acute taper angle.

To validate the simulated models, V15 and T90 were fabricated and tested by an EB dye. With interrogation lines referring to equivalent trajectories, each experimental gradient profile was compared with that retrieved from the corresponding model. The experimental and theoretical profiles exhibited comparable trends with 10% deviation. Without hardwired channel manifolds, diffusive mixing among stable laminas readily distributed a variety of concentrations along the V-shaped tapered microchannel.

In contrast to the constant diffusion dimension in T90, the ability to extend the gradient by V15 was due to the variable length of the diffusion dimension confined by channel geometry. Identical analyte molecules were forced to travel a longer distance to reach the interrogation line at d_{front} than dᵣₑₐᵣ as a result of the altered gradient profile at slow flow rates.

As to the laminar flow control by channel geometry and its effect on cell docking scalability, to incorporate biological cells within microdevice, a variety of cell localization approaches have been reported, including re-anchoring of trysinized adhesive cells after laminar flow patterning, trapping cells in hydrogel by selective photopolymerization, driving cells towards a stagnant zone in a T junction configuration, separating mother from daughter cell by cell scanning, as well as using enhanced dielectrophoretic traps. Among all these approaches, cell docking remains one of the easiest ways to handle large amounts of suspension cells with forgiving fabrication requirements. Driven by an adjustable docking flow, cell docking is capable of distributing a monolayer of cells on a localization structure (e.g., dam, weir or sandbag) parallel to the main flow route. Since the docking mechanism has been described previously, the present discussion is focused on how channel geometry affects docking capacity, i.e., the maximum quantity of single cells localizable on a sandbag component by cell docking.

Mathematical models for the V and T units have been developed to study cell docking in terms of docking potential, the liquid pressure difference across the sandbag component at the parallel line trajectories. Positive docking potential indicated cells were driven from the gradient component towards the corresponding sandbag position. For each microdevice unit, this potential was simulated in docking and gradient LLP conditions as shown in Table I above.

Although similar in gradient LLP condition, the docking potential profiles between V and T simulated units were markedly different at docking LLP. Originating from the channel geometric effect, variation of the simulated profiles was manifested in experiments as a docking capacity difference. A deeper study to this profile variation suggested why rear sandbag positions of the T unit were emptied in contrast to the fully occupied sandbag in the V unit. Initially one fold higher near the sandbag front, the docking potential in the T unit dropped below the value of the V unit at equivalent sandbag rear positions. It was the inability to sustain stable docking potential along the entire sandbag resulted in the compromised docking capacity in the T unit, even thought unoccupied sandbag positions were available. Further studies with increased docking potential were attempted in the T unit. However, squeezing through of cells near sandbag front positions was observed. All docked HL60 cells were subsequently stained by calcein-AM (CAM), a viable cell dye. The fluorescence resulted from the conversion of the non-florescent AM form into fluorescent calcein by nonspecific intracellular esterase in living cells in 10 minutes to demonstrate cell viability after docking event.

Under identical peripheral channel dimensions of the sandbag component as well as the LLP configuration, the scalability of docking capacity was still extensible by the channel geometries adjacent to the sandbag component. This enhanced docking capacity was a result of the local enlargement of the V-shaped channel that introduced a smaller pressure drop along both sides of the sandbag. Note that simplified models were developed to account for this phenomenon as described by the following equation:

$$Δ H = \frac{L}{{I}^{2}} {Q}^{′} \qquad ( 3 )$$

where H is the liquid level (Δ is a variety), L is the length of channel and Q′ is the flow quantity times a constant value related with fluid viscosity. For each model, the upper and lower channels are 10 units in length and share one common outlet. Assuming a high resistance sandbag component connecting the two channels so that negligible flow travels across channels, where a section of locally enlarged channel geometry (I=2 units) attributed to a small change in total channel resistance, all other sections have a unit cross-sectional dimension. In this scenario, it is clear that the difference in slope between plots corresponding to these simplified models is a result of the change of L As the enlarged geometry reduced the slopes between two channels, the docking potential is more stable along the sandbag. Interestingly enough, only a proportion of the liquid level difference is utilized for docking. In practice, one can set a relatively larger liquid level difference among vials to minimize pipetting errors, with such difference not being totally transferable to fragile mammalian cells.

To perform ideal cell docking in the subject microdevice, the docking potential should be stably distributed across the entire sandbag structure during docking, while being kept to a minimal value at gradient LLP. The stable docking potential over the sandbag resulted in full docking without squeezing and a minimal potential at gradient LLP, resulting in minimized disturbance to cells during the experiment.

### EXAMPLES

### Example 1

### Self-Delivery of Inhomogeneous Mixture Over Single Cells

With no increase in control complexity, high throughput bioassays were performed by the manipulation of laminar flow within the V-shaped channel. In addition, the throughput of the mixture preparation could also be improved with the leveraging of the diffusive mixing phenomenon. By using two analytes of different diffusion coefficients, superimposed characteristic gradient profiles could be generated and broadened simultaneously by the tapered V-shaped geometry. As a result, cells exposed to the superimposed gradients experienced a variety of inhomogeneous mixtures originating from a single homogeneous source. This approach offers opportunities to explore multiple analytes without complicated controls.

Although both are employed in viability assessment, CAM is expected to stain cytoplasm while TMR preferentially stains mitochondria. Of equal concentration, the heavier CAM dye (2.51×10⁻¹⁰ m²s⁻¹) was homogenized with the lighter TMR dye (4.79×10⁻¹⁰ m²s⁻¹) before delivery to the lower sandbag component. Simultaneously, the upper sandbag component was loaded with TMR alone. Since the dyes targeted different organelles, similar TMR response profiles were retrieved from both sandbag components to confirm negligible interference between the dyes employed.

From a single confocal micrograph, the pseudo-colored green (CAM), red (TMR) and combined images of cells after exposure to superimposed gradient profiles were used. For each dye, the normalized cell responses were plotted together with the associated dye gradient profile simulated in the V unit model. According to the simulated dye profiles, the lighter TMR was expected to be the dominate species at the sandbag front positions as it moved faster than CAM under identical flow rate. Good agreement was found in the combined image where cells were only stained in red (TMR) at the sandbag front. From right to left, green fluorescence was gradually intense in the cytoplasm of single cells until at the rear positions cells were stained in a similar fashion to equal CAM+TMR concentration.

From a homogeneous source, the microdevice generated a variety of inhomogeneous CAM+TMR concentrations that were automatically distributed to single cells over an extended sandbag component. It is possible to superimpose even more analyte without increasing control complexity, a great contrast to manually preparing analyte combinations with comparable diversity. Moreover, the compact design facilitated a single snapshot of all cells with high spatial resolution.

### Example 2

### Multiple Information Generated Form a High Throughput Single Cells Assay

High throughput dose-response and time-course single cells experiment were demonstrated by studying enzymatic cleavage of calcein-AM (CAM) in cytoplasm. Docked on one sandbag component, cells were exposed to a serial dilution of CAM with images captured at 1-minute intervals. As the microdevice was kept stationary throughout the experiment, images at various time points were perfectly aligned for subsequent GenePix analysis.

For a parallel-performed single experiment, a throughput of about 100 time-course CAM responses were retrieved from single cells along the chronological sequence of images while dose response information was derived from the identical series of cells along the sandbag component (entire length=2000 μm). Furthermore, the consistence of time-course response linearity between on-chip (90% docked cells with R²>0.95) and literature results in other cell lines as well as in our control experiment suggested equivalent bioassay environment was offered by the microdevice.

As a result of the high throughput design, the on-chip CAM kinetic profile was established by adequate data points while a similar trend was observed from results obtained in a conventional microplate reader. Albeit with larger fluctuation, the single on-chip experiment provided extra information regarding biological variability that was hidden in the composite microplate reader results. Whilst possible to be served as reference control, this biological variability was susceptible to I) percentage lethality in a population before treatment, II) different cell cycle stages, III) aging and IV) accumulation of genetic defects

A collection of test results, including a CAM time-course response, dose response and biological variability were performed in a single experiment executed in a high T/R V unit. Results retrieved in the microdevice were comparable to those in the literature and control experiments. Similarly, the compact design enabled a large number of cells to be interrogated at once, an important feature for high throughput kinetic analysis.

### Example 3

### Comparison of Parallel Experiments Performed on Compact Design Microdevice

It was the highly compact design of this microdevice that facilitated the comparative study between large amounts of cells on two sandbag components. Without laser irradiation, identical on-chip serial dilution of TMR (a xanthylium dye shows specific fluorescence staining of mitochondria by electrostatic interaction and staining fades out with loss of mitochondria membrane potential during apoptosis) was applied to incubate cells docked on both components. However, only a region of interest (ROI) on the upper sandbag was subjected to fast laser irradiation (˜70 ms/ROI of Helium-Neon laser) for 3 more minutes. A confocal image of both sandbags after the fast irradiation was developed.

Since replicated on-chip treatments were delivered to both sandbag components but enhanced TMR response was only exhibited on the upper sandbag, there was a strong correlation between off-chip fast irradiation and the enhanced response. Furthermore, despite the large ROI coverage over the upper sandbag, the enhanced response was evidenced for the cells incubated at higher TMR concentration near the sandbag rear. Therefore, the conclusion is that the enhanced response was at least related to 1) concentration of TMR and 2) the amount of laser irradiation. An EC50 value of 3.73 μM was estimated to describe the TMR concentration required for this response enhancement.

Inevitably, laser irradiation required for capturing the final image might complicate result interpretation. Fortunately, the enhanced effect originated from the fast irradiation could still be resolved by comparing the results with the responses obtained from the lower sandbag with a corresponding set of internal controls. In the meantime, the compact microdevice design resulted in doing parallel experiments on both components, providing highly comparable results.

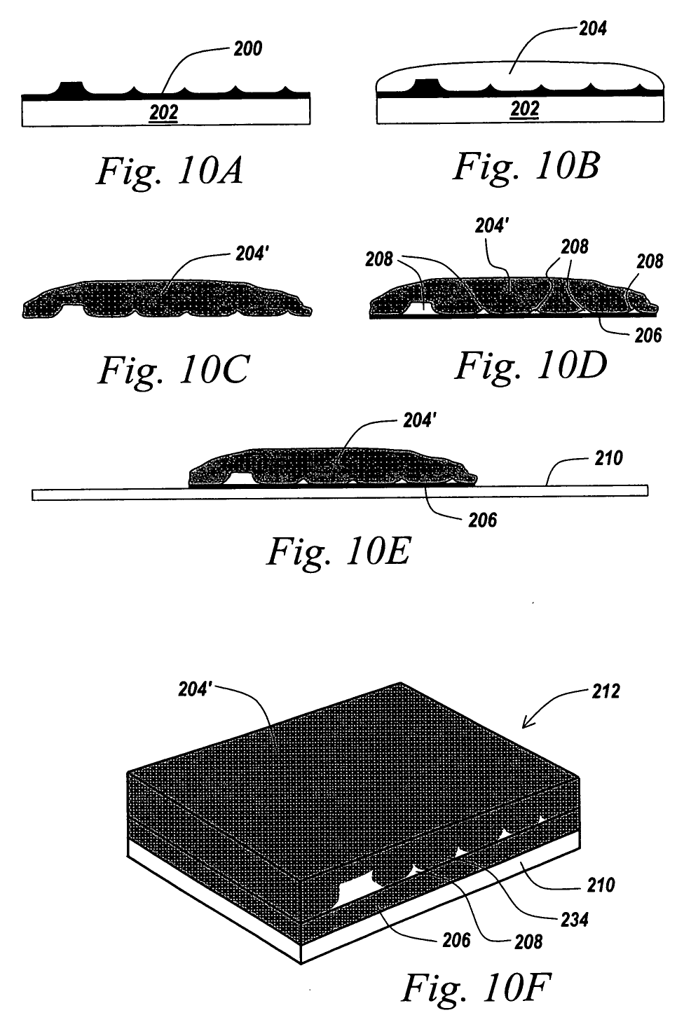

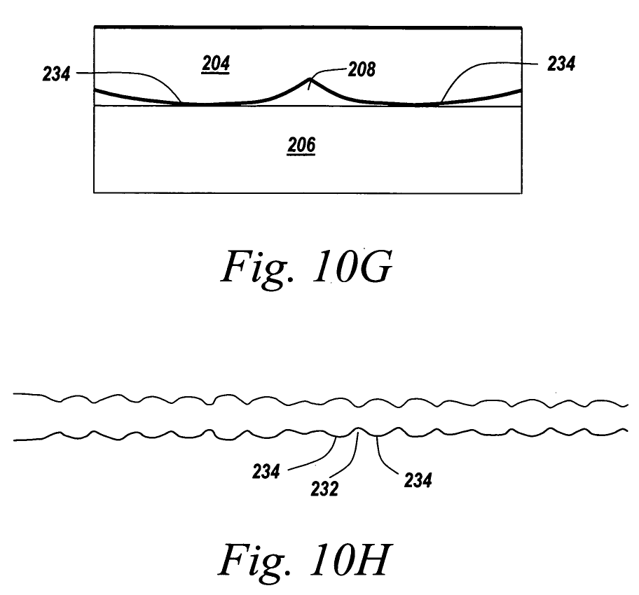

Referring now to FIGS. 10A-10H, what is shown is a step-by-step procedure for forming the microchannels in the aforementioned sandbag structure.

Referring to FIG. 10A, a positive copper relief 200 is formed over a substrate 202. This copper positive relief is called a master.

As seen in FIG. 10B, a PDMS viscous solution 204 is poured over the master and is cured until it solidifies.

As shown in FIG. 10C, the cured PDMS solution 204′ is peeled off the master such that the peeled-off PDMS is a solidified negative relief.

As seen in FIG. 10D, the solidified negative relief PDMS 204′ is sealed against a flat PDMS 206. The sealing of the negative PDMS replica against the flat PDMS results in channels 208 between the solidified negative relief PDMS and the flat PDMS.

Referring to FIG. 10E, the flat PDMS 206 carrying the solidified negative relief PDMS 204′ is placed on a glass slide 210. This is because, though solidified, PDMS 204′ is still rather soft. Thus, the glass slide makes it easier to hold the FIG. 10E structure.

Referring to FIG. 1OF, one sees a isometric view of the trimmed PDMS sandwich 212 illustrating the cured PDMS 204′ on top of the flat PDMS 206, which is in turn on top of glass plate 210.

As can be seen, the microtunnel structure is provided by channels 208. Thus, as can be seen in FIG. 10H, one has a sandbag structure in which microchannels 232 exist between adjacent domed structures 234, with the structure in FIG. 10G illustrating the microchannel formation from a side view perspective.

It is the interstices between adjacent domes that provides for the subject microchannel structure that permits cell docking.

It is a feature of the tapered construction that the gradient flow produces a gradient that tapers off from a maximum at the input end of the device to the outlet. The shape of the gradient curve depends on the input volume, with the larger input volume providing a more pronounced gradient tapering. Moreover, it can be shown that when docking potential is graphed against sandbag position, the ΔP is relatively linear.

Referring now to FIG. 11, what is shown is how the diffusion or reaction distance, d, that defines the gradient is measured in the tapered channel in which two fluids are introduced at the inlet end, with the taper being 15° in this case. Here it can be seen that d_{front}>dᵣₑₐᵣ.

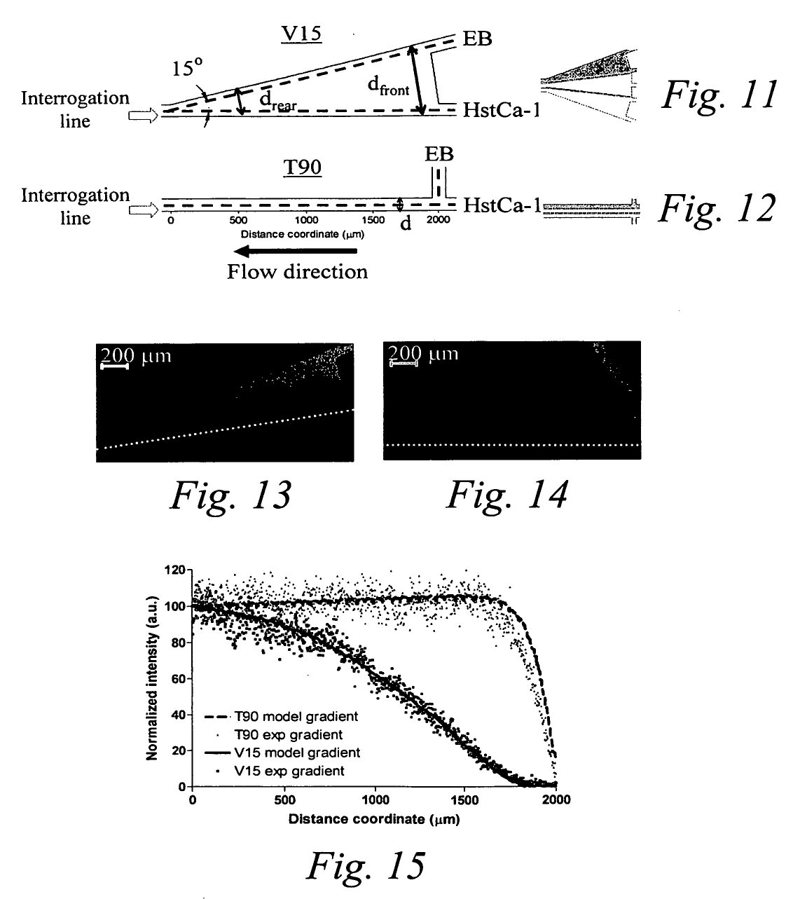

FIG. 12 is a conventional T-type non-tapered channel in which the two fluids are introduced orthogonally at the inlet end and, with the diffusion reaction distance d being constant and that associated with the inlet end of the channel.

FIG. 13 is a photomicrograph showing the diffusion gradient along the length of the sandbag structure from inlet end to outlet end, whereas FIG. 14 is a photomicrograph showing that when using a non-tapered channel, all of the diffusion takes place at the inlet end, thus precluding different concentrations.

FIG. 15 is a graph illustrating that there is a model gradient expected from a tapered channel with a 15° taper and that the experimental gradient follows the theoretical model, also showing that there is no gradient in the conventional T-channel non-tapered systems, with the tapered gradient providing the ability to subject cells to different concentrations or differing dosages utilizing a single sample source and a single source of reactant.

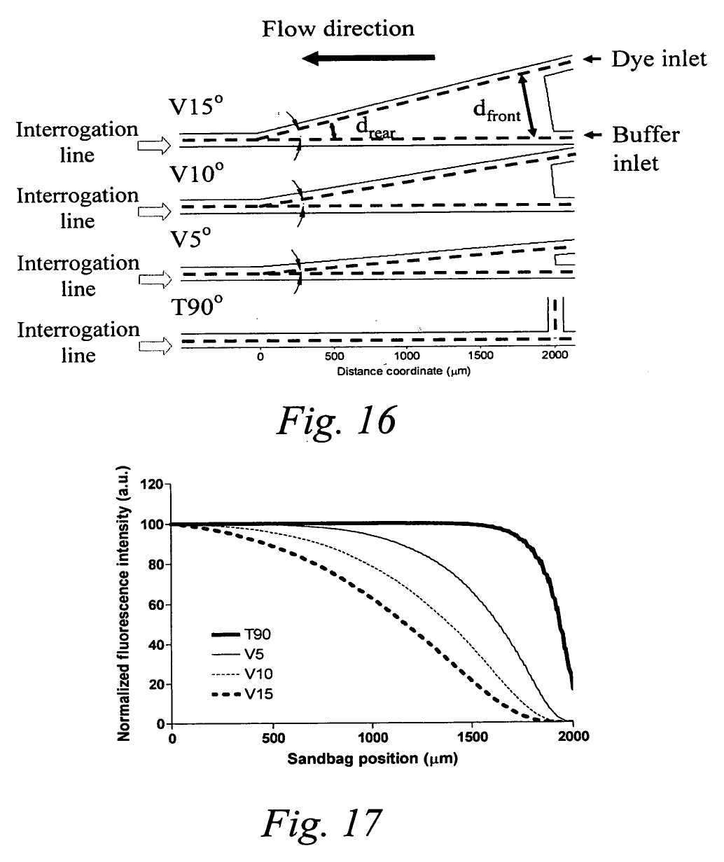

FIG. 16 is a diagrammatic illustration of the use of differing taper angles, with the taper being in the flow direction in which two fluids, namely a dye and a buffer, are introduced at the inlet end, with the diffusion distance being maximal at the input end and decreasing toward the outlet end, also showing that for the conventional T-channel there is no diffusion distance change along the length of the channel.

FIG. 17 is a graph of normalized fluorescence intensity versus sandbag position, confirming the different gradients for different taper angles, showing confirmation of the diffusion gradient controls the reaction between two fluids as they react down the channel.

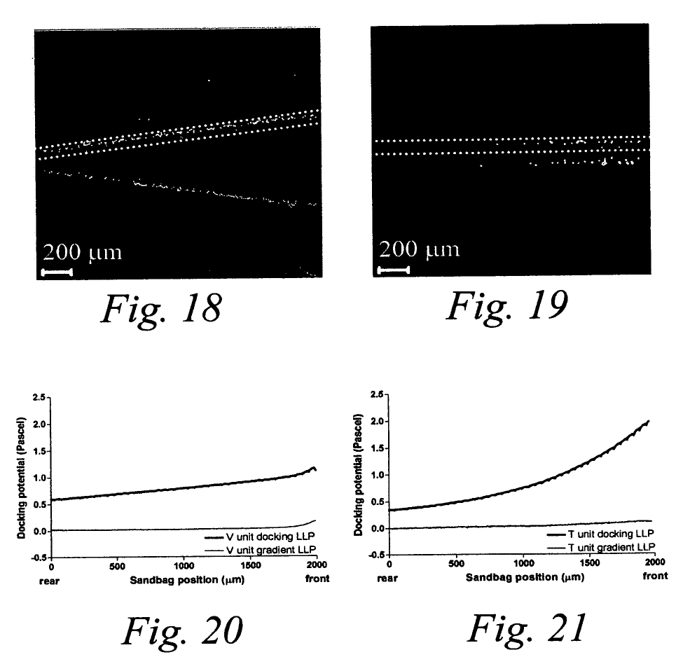

FIG. 18 is a photomicrograph illustrating the docking of cells along the sandbag for one of the two sandbag structures in a three-channel embodiment, whereas FIG. 19 shows cell docking for the conventional T-channel.

Referring to FIG. 20, the docking potential for cells at various sandbag positions is shown. Here it can be seen that the docking potential depends upon the gradient and that a linear relationship exists so that one could ascertain that one has one cell per sandbag interstice, thus to permit calculating a predetermined gradient will result in a linear docking potential with position.

On the other hand, as illustrated in FIG. 21, for a conventional T-channel, while the docking gradient is linear, the docking potential is not linear, thereby adding variability to the results for the conventional T-channel systems due to the lack of predictability of the number of cells at any given position, assuming a sandbag is used in the T-channel configuration.

Although similar in gradient LLP condition, the docking potential profiles between V and T simulated units were markedly different at docking LLP. Originating from the channel geometric effect, variation of the simulated profiles was manifested in experiments as a docking capacity difference. A deeper study into this profile variation suggested why rear sandbag positions of the T unit were emptied in contrast to the fully occupied sandbag in the V unit. Initially one fold higher near the sandbag front, the docking potential in the T unit dropped below the value of the V unit at equivalent sandbag rear positions. It was the inability to sustain stable docking potential along the entire sandbag that resulted in the compromised docking capacity in the T unit, even though unoccupied sandbag positions were available. Further studies with increased docking potential were attempted in the T unit. However, squeezing through of cells near sandbag front positions was observed. All docked HL60 cells were subsequently stained by calcein-AM (CAM), a viable cell dye. The fluorescence resulted from the conversion of the non-florescent AM form into fluorescent calcein by nonspecific intracellular esterase in living cells in 10 minutes to demonstrate cell viability after the docking event. The effect of geometry observed in FIGS. 18-21 is further elucidated by a simplified model as illustrated in FIGS. 22-26.

Referring now to FIG. 22, what is shown is a structural model comprising upper and lower microchannels interconnected with a “sandbag-like” structure, sandbag component. The two channels were of circular cross-sectional dimension and unit length. The sandbag components, positioned at the midway of the channel system, consisted of microtunnels that allowed cells to dock individually at positions along the channel. Gradient and docking components were introduced into two channels adjacent to the sandbag component with channel radii r_{c}=1. Applied pressure was 1-fold higher to the upper (1 unit pressure) than the lower channel inlet (0.5 unit pressure) while flows were converged to a common outlet at zero pressure. Thus, a right-to-left fluid flow and a pressure difference were generated along and between the two channels respectively. The sandbag component was assumed to have high fluidic resistance such that negligible flow could pass through. Under these conditions, a significant pressure drop that shows a linear relationship with respect to the length along both microchannels resulted. Owing to the different rates of pressure drop within the two microchannels, the resultant pressure differences across microchannels (ΔP_{b}′) are unstable adjacent to sandbag component.

Referring now to FIG. 23, what is shown is a modified structural model compared to that depicted in FIG. 22. In this model, the channel radii of gradient and docking components are enlarged such that, r_{c}=2. In accordance with Equation 1 mentioned above, increasing radius could proportionally reduce the ΔPₐ′ values, leading to the resultant ΔP_{b}′ values decreased less steeply for r_{c}=2 than r_{c}=1. Therefore, a stable and constant pressure difference can be achieved by modulating the slope of pressure profiles with a 1-fold radii increment at the gradient and docking components.

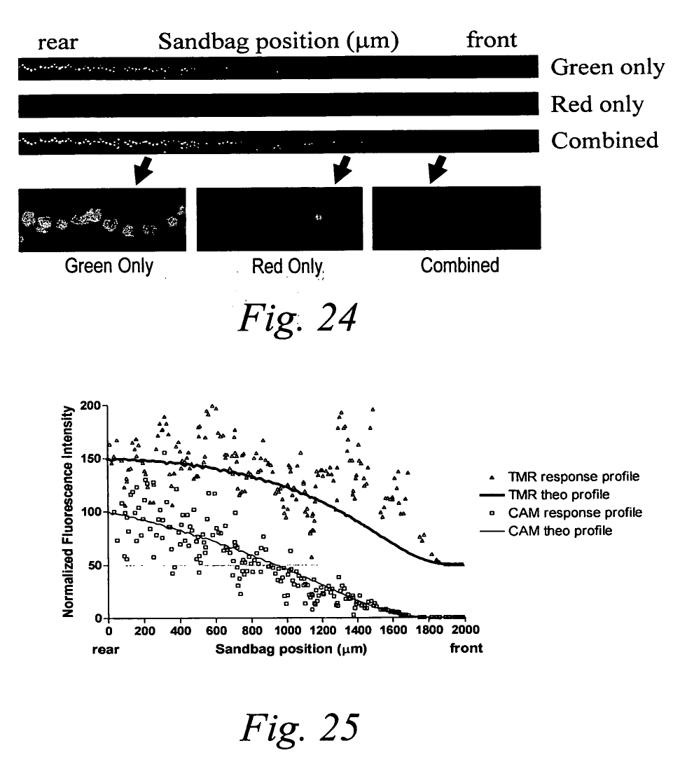

Referring now to FIG. 24, utilizing Calcein-AM one can see that cells are stained in green and with respect to sandbag position, the fluorescence intensity of the stain decreases from rear to front, which is the same when detecting the TMR stain (in red).

When these results are combined, between the rear and front of the structure, one can see the green concentrations at the left, which go to the green-red concentrations in the middle and the red concentrations on the right-hand side. This shows simultaneous staining of cells under two different continuum of two different dyes. As the two dyes target different organelles, mitochondria (red stain by TMR) and cytoplasm (green stain by CAM) are clearly resolved under one single confocal image.

Correspondingly and referring now to FIG. 25, when graphing normalized fluorescent intensity versus sandbag position, both the theoretical TMR profile and the theoretical CAM profile correspond quite nicely to the experimental response profiles for TMR and CAM respectively. This graph explains why the TMR stain is dominant at the front of sandbag positions. Due to the lower molecular weight of TMR when compared to CAM, this lighter dye diffuses faster and arrives to sandbag front positions at higher concentration.

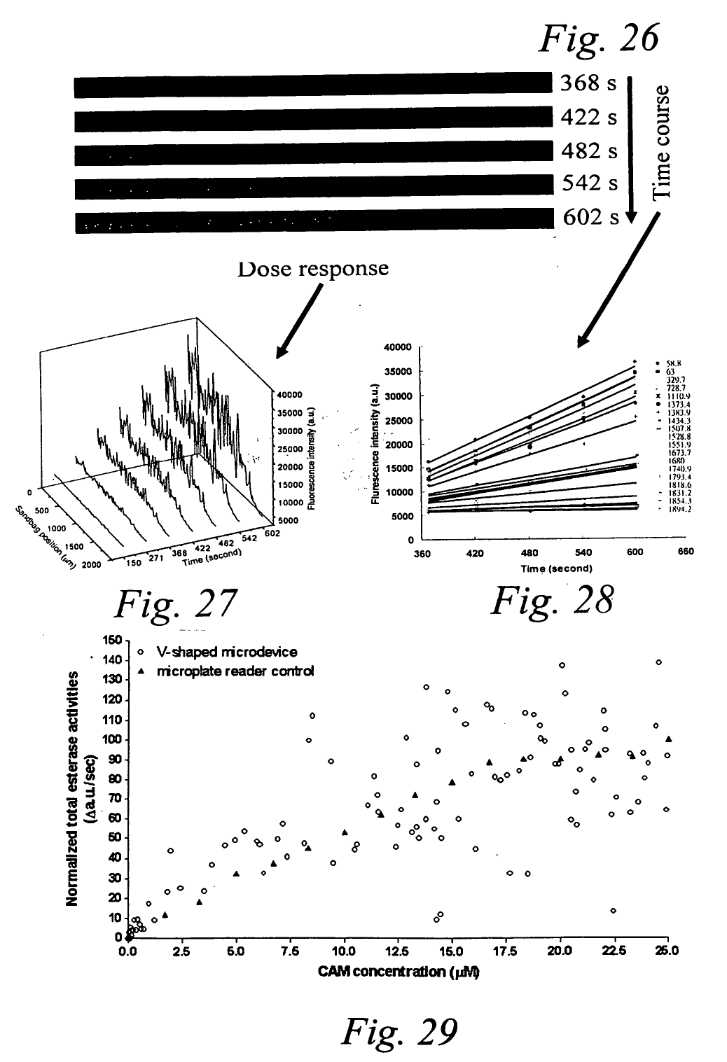

Referring to FIG. 26, taking five confocal images, one can see the time course response of each individual cell while from each image, the dose response of the cells under differing CAM concentrations can be retrieved along the sandbag. The 5 dose response profiles were plotted in dimension in FIG. 27, while the time course of selected cells was plotted in FIG. 28.

The graph of FIG. 28 graphs fluorescence intensity over time and shows the linear relationship of single cells response over time when exposed to differing CAM concentrations. CAM response linearity is consistent to literature findings.

Referring to the graph of FIG. 29, when graphing a normalized esterase activity against CAM concentrations, one can see the enzyme kinetic plot. A control experiment was performed by a conventional microplate reader to compare the results with microchip results. Although data were scattered in microchip result, the data trend and kinetic parameters obtained both methods were comparable.

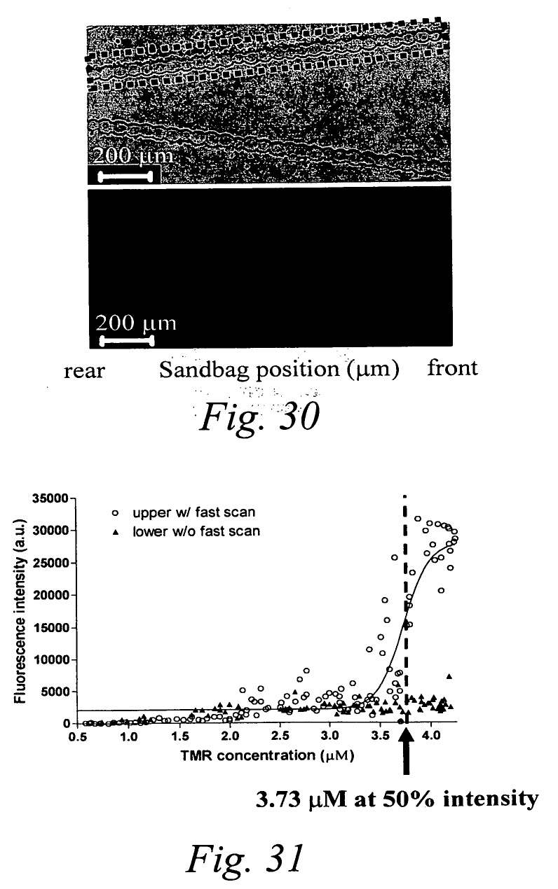

Referring to FIG. 30, photomicrographs are shown one on top of the other, with the top photomicrograph showing cells docked on both sandbags but only those encircled by the black dotted framework are subjected to laser irradiation. The bottom photomicrograph shows the response of cells after the experiment in which cells on both sandbags were exposed to differing TMR concentrations, but with laser irradiation exclusively applied to cells docked on the upper sandbag. The purpose of these photomicrographs is to show the power of the microchip to perform rapid analysis and retrieve effective concentration of a photodynamic therapy drug candidate that is sensitive to light irradiation.

The response profiles of cells docked on the upper and lower sandbags of FIG. 30 are plotted in FIG. 31. Here, it can be seen that fluorescence intensity as graphed against TMR concentration reveals that enhanced fluorescence response is observed when cells are exposed to 1) fast scan laser irradiation and 2) relatively high TMR concentration. Note, a significantly enhanced response is observed. Note further that there is an average peak in intensity at a 3.73 μM at 50% intensity. This indicates the microchip can easily retrieve the 50% effective concentration (EC50) of TMR that is required to trigger the observed fluorescence enhancement in HL60 cells.

In summary, the tapered microchannel structure allows individual cells to be reacted with a continuum of concentrations or dosages of an analyte or drug from one sample, the concentrations depending upon the linear gradient that is established by the tapered structure.

While the present invention has been described in connection with the preferred embodiments of the various figures, it is to be understood that other similar embodiments may be used or modifications or additions may be made to the described embodiment for performing the same function of the present invention without deviating therefrom. Therefore, the present invention should not be limited to any single embodiment, but rather construed in breadth and scope in accordance with the recitation of the appended claims.

What is claimed is:

1. A method for on-chip monitoring of cellular reactions, comprising the step of:
subjecting cells isolated in a sandbag structure to different concentrations of analytes or drugs in a one-step operation involving only one sample of analyte or drug at one concentration.

2. The method of claim 1, wherein the step of subjecting the cells to different concentrations includes providing a microfluidic device in which two fluids laminarly interact based on a continuum of concentrations, the continuum of concentrations being formed by tapering the laminar flow interaction channel such that as the two laminar fluid streams flow from a wide inlet to a narrowed outlet, the diffusion between the two flows increasing as the two fluids are forced together by the narrowing walls of the interaction channel.

3. The method of claim 2, wherein individual cells for which reaction is sought are carried at various locations in a sandbag structure, the sandbag structure forming one side of the tapered channel.

4. The method of claim 3, wherein the tapered channel subjects the individual cells along the sandbag structure to different concentrations.

5. The method of claim 4, wherein the diffusion at the inlet end of the tapered channel is lower than the diffusion at the outlet end, thus to provide the diffusion continuum.

6. The method of claim 5, wherein the increased diffusion towards the outlet end of the tapered channel provides a continually increasing concentration as the two streams are squeezed together by the tapered interaction channel.

7. The method of claim 1, wherein different concentrations occur at different positions relative to the sandbag structure such that individual cells at different locations in the sandbag structure are subjected to different concentrations of analytes or different dosages of drugs, whereby a cell line can be subjected to a continuum of concentrations, thereby to provide a continuum of test results based on different concentrations in one step.

8. The method of claim 7, wherein the cell interaction is read out at different positions on the sandbag structure, the reaction at different positions correlated to the specific concentration or dosage at the particular position at which the interaction is read out.

9. The method of claim 1, and further including the step of positioning cells from a fluid stream at different positions along the sandbag structure and immobilizing the cells in the sandbag structure after being positioned.

10. The method of claim 2, wherein the subjecting step includes reacting the individual cells at different positions with different concentrations due to the different and increasing diffusions as one proceeds to the outlet end of the tapered interaction channel.

11. The method of claim 10, wherein only one concentration or dosage of analyte or drug is used at the inlet end of the tapered interaction channel, thus to permit testing at a virtual continuum of concentrations or dosages from one sample at one concentration without having to prepare different samples at different concentrations or dosages.

12. The method of claim 1, wherein the increasing diffusions with distance from the inlet end define a concentration gradient and wherein the concentration gradient corresponds to the difference in interaction channel width transverse to the flow direction from the inlet to the outlet, the variation in width constituting a dimensional gradient, with the longer the dimensional gradient, the longer the region in the sandbag structure subjected to discernibly different concentrations.

13. The method of claim 1, wherein the tapered interaction channel is divided into thirds by two linear sandbag structures that form dividers between the three channel chambers.

14. The method of claim 13, wherein the two sandbag structures are separated at the inlet end and are tapered towards a common outlet end.

15. The method of claim 14, and further including the steps of simultaneously testing normal cells and non-normal cells with the same analytes or drugs at the same concentrations or dosages by docking normal cells on one of the sandbag structures and non-normal cells on the other of the sandbag structures, thus to permit comparison of the interaction of the two cell types with the same sample at the same concentration or dosage.

16. The method of claim 15, and further including the step of adjusting fluid pressure to determine which side of the sandbag structure the cells lie on.

17. The method of claim 16, and further including the steps of injecting the analyte or drug into the middle channel and simultaneously testing normal cells on the inside of one sandbag structure with the analyte or drug while simultaneously testing non-normal cells on the inside of the other sandbag structure with the same drug or analyte at the same dosage or concentration.

18. The method of claim 2, wherein fluid pressure control is determined by the volume of liquid introduced into the tapered interaction channel.

19. The method of claim 18, and further including the use of a dual sandbag structure and the step of simultaneously running two parallel experiments in which cells are positioned using the liquid volume sample pressure control method.

20. The method of claim 1, and further including the step of adjusting the concentration profile by using low fluidic flow rates and passive fluidic controls.

21. The method of claim 20, wherein the passive fluidic controls include the step of controlling fluid pressure through the volume of liquid introduced into the tapered interaction channel.

22. A microchannel chip for monitoring cellular reactions, comprising:
a body having a tapered interaction channel, tapered from an inlet end to an outlet end;
a sandbag structure to one side of said tapered interaction channel adapted to receive single cells in the interstices thereof; and,
a liquid under pressure introduced at the wide inlet end of said channel to interact with the cells in said sandbag structure so as to subject said cells to differing diffusion rates and consequent concentrations of said liquid.

23. The microchannel chip of claim 22, and further including a fluorescence measuring instrument for measuring the fluorescence of cells at different positions on said sandbag structure, thus to ascertain the reaction of said cells to said liquid at differing concentrations based on the position along said sandbag structure at which said measurement is taken.

24. The microchannel chip of claim 22, wherein said microchannel structure includes three tapered channels, including first and second tapered outer channels; a first sandbag structure forming a divider at one side of said first tapered outer channel; a tapered central channel bordered on one side by said first sandbag structure; a second sandbag structure to the other side of said central channel, said second tapered outer channel formed on one side by said second sandbag structure.
